(19) United States
(12) Patent Application Publication
(10) Pub. No.: US 2004/0076684 A1
(43) Pub. Date: Apr. 22, 2004

(54) Nutritional compositions for alleviating fatigue

(76) Inventor: Ching-Chi Lin, Shijr City (TW)

Correspondence Address: TROXELL LAW OFFICE PLLC, 5205 Leesrurg Pike, Suite 1404, Falls Church, VA, 22041, US

(21) Appl. No.: 10/272,904

(22) Filed: Oct. 18, 2002

(51) Int. Cl. A61K 35/32; A61K 35/78

(52) U.S. Cl. 424/549; 424/728; 424/777

(57) ABSTRACT

A nutritional compositions for alleviating fatigue is produced by grinding essentially Panax ginseng, androgen-containing Cervus sika, vitamin A- and B-containing extract of fruit meat of Euphoria longana, Dioscorea batalus, and Acanthopanax senticosus into powder, and then mixing and formulating the resulting powders with gelatin and pure water into a formulation, wherein, in 350 mg of said nutritional composition, the effective amounts of each components is: Panax ginseng 105 mg, androgen extracted from Cervus sika 51 mg, Acanthopanax senticosus 15 mg, gelatin 86 mg, pure water 14 mg, and vitamin A and B extracted from fruit meat of Euphoria longana 24 mg each.

### BACKGROUND OF THE INVENTION

1. Field of the Invention

The invention relates to a nutritional compositions for alleviating fatigue, and, in particular, to a nutritional compositions for alleviating fatigue having effects of nourishing and strengthening the body as well as restoring from fatigue.

2. Description of the Prior Art

Being in a modern busy and highly stress society, due to heavy burdens or miscellaneous business, people often works at night while sleep at day. Further, instead of taking meal at normal time, frequently too much eating and drinking always take places for social intercourse and dining out. Moreover, insufficient sport capacity results into poor blood circulation and/or diseases as well as mental disorder. All of these phenomena lead modern people to a poor body state that, not only a long-term fatigue feeling persists, but also cannot restore from resting. These further affect deeply into the individual metal aspect that leads men having a heart but no strength in facing anything. A variety of commercially available oral pharmaceuticals that were claimed to be capable of eliminating fatigue had been proposed currently in order to solve the above-described situations. Unfortunately, most of these pharmaceuticals were based on drugs synthesized from chemical components such that long-term administration thereof would result readily into one's addiction as well as hypersensitivity. Accordingly, these types of pharmaceuticals are not amenable to be administrated by all people.

In view of the foregoing, current so-called fatigue-restoring pharmaceutical products are not of perfect design but needing improvement.

The inventor of this application has been studied intensively in an effort for solving the above-mentioned disadvantages associated with the prior art, and finally, found successfully that a nutritional composition having effects of alleviating fatigue and restoring strength without above-indicated drawbacks associated with the prior art is satisfactorily meet the object of the invention and thus accomplishes the invention.

### SUMMARY OF THE INVENTION

Accordingly, the object of the invention is to provide a nutritional composition for alleviating fatigue, characterized that it is based on the pathology of traditional Chinese medicine and formulated with natural Chinese herbal materials without any chemically synthesized drugs and hence will not give any adverse effect on human body.

Another object of the invention is to provide a nutritional composition for nourishing and strengthening human body as well as restoring from fatigue.

### DETAILED DISCRIPTION OF THE PREFERRED EMBODIMENTS

Nutritional composition that can achieve the above-mentioned objects of the invention comprises, in specified amount, Panax ginseng C. A. Mey, androgen extracted from Cervus sika Temm. vitamin A and B both extracted from fruit meat of Euphoria longana Lam, Dioscorea batalus Decne, Acanthopanax senticosus (Ruper Et Maxim), gelatin and pure water.

The nutritional composition for alleviating fatigue provided according to the invention is formulated with natural Chinese medicinal materials, and comprises essentially Panax ginseng C. A. Mey, androgen extracted from Cervus sika Temm, vitamin A and B extracted from fruit meat of Euphoria longana Lam, Dioscorea batalus Decne, Acanthopanax senticosus (Ruper Et Maxim), gelatin and pure water. Among essential components of the nutritional composition for alleviating fatigue provided according to the invention, Panax ginseng C. A. Mey, Cervus sika Temm., fruit meat of Euphoria longana Lam, Dioscorea batalus Decne, and Acanthopanax senticosus (Ruper Et Maxim) are all Chinese medicinal materials. Among them, Panax ginseng has effects of anti-fatigue, enhancing immunity, strengthening heart and anti-allergy; Cervus sika has effects of eliminating fatigue, strengthening spirit, promoting function of stomach and intestine, as well as treating headache, dizzy, tinnitus, anaphrodisia, weak lumbar and knee; Dioscorea batalus can be used as nourishing, strengthening and anti-diarrhea drugs, as well as can treat anorexia, fatigue, weak stomach and intestine and diabetes mellitus; fruit meat of Euphoria longana has a hematinic and ataractic effect, and is suitable for anemia, palpitate, insomnia and obliviscence; and Acanthopanax senticosus has effects of anti-rheumatic, strengthening sinew, activating blood, and decongestion, and is suitable for treating anti-chill and wet paralysis, sinew contracture, lumbago, anaphrodisia, weak foot and beriberi, botch, sphacelus, neoplasm, poison, hurt from falling, fighting and laboring. From description above, it can be suggested that materials used in the nutritional compositions for alleviating fatigue according to the invention have effects of anti-fatigue, strengthening spirit, nourishing, analeptic, sinew strengthening, and the like. Thus, the nutritional composition produced by grinding Panax ginseng, Cervus sika, fruit meat of Euphoria longana, Dioscorea batalus, and Acanthopanax senticosus into powder followed by adding gelatin and pure water and formulating can indeed achieve the effect of nourishing, analeptic and anti-fatigue.

For convenient administration, the nutritional composition according to the invention can be formulated as dose unit such as capsules which contains the formulation in a specified amount of, for example, 350 mg. In order to achieve the effect of nourishing, analeptic and anti-fatigue, contents of various essential components in the nutritional composition can be varied. In a preferred embodiment, the nutritional composition has a composition as follows: 105 mg of Panax ginseng, 51 mg of androgen extracted from Cervus sika, 45 mg of Dioscorea batalus, 15 mg of Acanthopanax senticosus, 86 mg of gelatin, 14 mg of pure water, and 24 mg each of vitamin A and B extracted from fruit meat of Euphoria longana.

### Features and Effects of the Invention

The nutritional composition according to the invention has several advantages over products of prior art as follow:

The nutritional composition according to the invention is formulated by employing substantially natural herbal materials, i.e., Panax ginseng, Cervus sika, fruit meat of Euphoria longana, Dioscorea batalus, and Acanthopanax senticosus, and extract from animal, i.e., gelatin, without any chemically synthesized drug, and hence can avoid problems as drug addiction or allergy.

By virtue of effects of anti-fatigue, strengthening spirit, nourishing, analeptic, sinew strengthening, and the like derived from Panax ginseng, Cervus sika, fruit meat of Euphoria longana, Dioscorea batalus, and Acanthopanax senticosus, through formulating with gelatin and pure water, the nutritional composition according to the invention thus produced can exhibit effects of nourishing, analeptic and anti-fatigue.

Many changes and modifications in the above-described embodiments of the invention can, or course, be carried without departing from the scope thereof. Accordingly, to promote the progress in science and the useful arts, the invention is disclosed and is intended to be limited only by the scope of the appended claims.

What is claimed is:

1. A nutritional compositions for alleviating fatigue, characterized in that is produced by grinding essentially Panax ginseng, androgen extracted from Cervus sika, vitamin A and B both extracted from fruit meat of Euphoria longana, Dioscorea batalus, and Acanthopanax senticosus into powder, and then mixing and formulating the resulting powder with gelatin and pure water into a formulation.

2. A nutritional compositions for alleviating fatigue as recited in claim 1, wherein the effective amount of Panax ginseng is 105 mg in 350 mg of said nutritional composition.

3. A nutritional compositions for alleviating fatigue as recited in claim 1, wherein the effective amount of androgen extracted from Cervus sika is 51 mg in 350 mg of said nutritional composition.

4. A nutritional compositions for alleviating fatigue as recited in claim 1, wherein the effective amount of vitamin A extracted from fruit meat of Euphoria longana is 24 mg in 350 mg of said nutritional composition.

5. A nutritional compositions for alleviating fatigue as recited in claim 1, wherein, the effective amount of vitamin B extracted from fruit meat of Euphoria longana is 24 mg in 350 mg of said nutritional composition.

6. A nutritional compositions for alleviating fatigue as recited in claim 1, wherein the effective amount of Dioscorea batalus is 45 mg in 350 mg of said nutritional composition.

7. A nutritional compositions for alleviating fatigue as recited in claim 1, wherein the effective amount of Acanthopanax senticosus is 15 mg in 350 mg of said nutritional composition.

8. A nutritional compositions for alleviating fatigue as recited in claim 1, wherein the effective amount of gelatin is 86 mg in 350 mg of said nutritional composition.

9. A nutritional compositions for alleviating fatigue as recited in claim 1, wherein the effective amount of pure water is 14 mg in 350 mg of said nutritional composition.
